(19) United States
(12) Patent Application Publication
(10) Pub. No.: US 2024/0350765 A1
(43) Pub. Date: Oct. 24, 2024

(54) AROMATHERAPY CARRIER AND PACKAGING

(71) Applicant: ORLANDO HEALTH, INC., Orlando, FL (US)

(72) Inventors: Keely DeGroot, Orlando, FL (US); Anthony Mango, Orlando, FL (US); Alison Ruiz, Orlando, FL (US); Kelly Kollstedt, Orlando, FL (US); Jennifer Wittenberg, Orlando, FL (US); Ellen Reising, Orlando, FL (US); Curtis Chadderdon, Twin Falls, ID (US); Joanne Pizzo, Twin Falls, ID (US)

(73) Assignee: ORLANDO HEALTH, INC., Orlando, FL (US)

(21) Appl. No.: 18/639,376

(22) Filed: Apr. 18, 2024

Related U.S. Application Data
(60) Provisional application No. 63/497,327, filed on Apr. 20, 2023.

(51) Int. Cl. A61M 21/02; A61M 21/00

(52) U.S. Cl. CPC A61M 21/02; A61M 2021/0016

(57) ABSTRACT

An aromatherapy product may have an aromatherapy oil configured for releasing an aroma when exposed to an outside environment. The aromatherapy product also may include a carrier, the carrier having an absorption portion for receiving a predetermined amount of the aromatherapy oil, where the absorption portion includes a wood-based fabric. The wood-based fabric may include, and in some cases may consist of, bamboo. The aromatherapy product may be individually packaged.

### CROSS-REFERENCE TO RELATED APPLICATION

This application claims priority to U.S. Provisional Application No. 63/497,327, filed on Apr. 20, 2023, the entirety of which is hereby fully incorporated by reference herein.

### TECHNICAL FIELD

The invention relates to the field of medical devices, such as carriers for aromatherapy oils and related packaging devices and methods.

### BACKGROUND

Aromatherapy is a therapeutic procedure where aromatic materials, such as essential oils and other aroma compounds, are used to improve a human's psychological or physical state, including as a comfort aid. For example, in a medical setting, practitioners may employ aromatherapy to treat anxiety, depression, nausea, insomnia, appetite issues, dry mouth, and a variety of other human conditions. As a comfort aid and/or for cosmetic use, aromatherapy may be used to cleanse the body. Existing aromatherapy oils typically include a mixture of aroma-providing essential oils along with a carrier oil, such as olive oil.

A variety of aromatherapy treatment options are available. For example, several carrier products are available, such as candles, wristbands, patches, necklaces, keychains, etc., that have an absorbent material filled with the aromatherapy oil. These products typically utilize a low-concentration essential oil mixture with a low rate of diffusion such that they last for a long period of time. In medical applications, certain options include dropping a few drops of a concentrated essential oil directly massaging the aromatherapy oil into the patient's skin, which may provide prolonged, uninterrupted exposure as the aromatherapy oil diffuses.

Currently-available aromatherapy products have several deficiencies. For example, current aromatherapy oils, mixed at their ideal strength, are aromatically volatile and can affect other people in the vicinity of the patient, and also diffuse extremely quickly (meaning storage is an issue). However, in products where concentration is reduced, the oils are slow to diffuse, meaning surrounding air may be affected for a period of time that is longer than optimal. Refillable products have been used to address some of these issues, but such products require additional handling of essential oils, and refilling with precise quantities of the aromatherapy may be challenging. Finally, since available products for carrying the oil are typically disposed of in the patient's trash, these products may end up at a landfill causing hazards to workers and other people at that location.

In view of this background, improved aromatherapy carriers are desirable, along with improved packaging.

### BRIEF DESCRIPTION OF THE DRAWINGS

The present embodiments are illustrated by way of example, and not by way of limitation, in the figures of the accompanying drawings. The following figures are illustrative only, and are not intended to be limiting.

FIG. 1 is an illustration generally showing an aromatherapy carrier in accordance with certain aspects of the present disclosure.

FIG. 2 is an illustration showing an aromatherapy carrier having separate absorbent and non-absorbent portions in accordance with certain aspects of the present disclosure.

FIG. 3 is an illustration showing packaging of an aromatherapy carrier in accordance with certain aspects of the present disclosure.

### DETAILED DESCRIPTION

In view of the background above, the present disclosure describes improved embodiments of aromatherapy carriers and related packaging, which have several advantages over existing devices. For example, the carriers described herein may be formed of an absorbent bamboo fabric or other wood-based fabric, which have surprisingly provided an improved aromatherapy carrier based on their particular absorbent properties along with their biodegradability. Without limitation, other improvements provided by the present embodiments include specific portion-sizing based on characteristics of the carrier (and packaging), thereby removing volatile oils from unwanted environments.

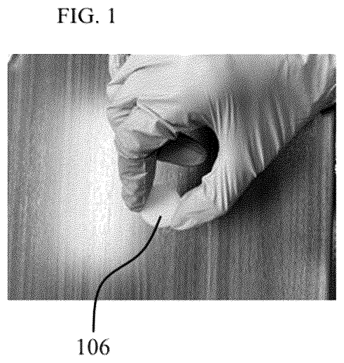

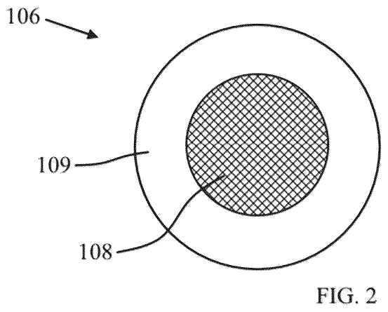

Referring now to the figures, FIG. 1 shows an aromatherapy system generally including an aromatherapy oil that is configured for releasing an aroma when exposed to an outside environment. The aromatherapy oil is at least partially held in place within a carrier 106. E.g., the carrier 106 may have an absorption portion 108 that carries the aromatherapy oil via absorption. In some embodiments (e.g. shown in FIG. 2), the absorbent portion 108 may be surrounded by, or otherwise adjacent to, a non-absorbent portion 109, which may be advantageous for providing a particular shape or structure to the carrier 106 where the absorbent portion 108 does not have sufficient rigidity for shape retention, but this is an optional feature.

The carrier 106 may be formed of any suitable material. In certain embodiments, at least the absorbent portion 108 of the carrier 106 is formed with a wood-based fabric, such as a bamboo-fiber fabric. Optionally, the absorbent portion 108, and perhaps the entire carrier 106, may substantially exclude other materials such that the absorbent portion 108 or full carrier 106 consists of the wood-based fabric.

Using a wood-based fabric to form the carrier 106 is associated with several advantages. The bamboo may act as a diffuser for the aromatherapy oil, which may facilitate light oil dispersion into the air once the packet is opened. For example, bamboo fibers are naturally resistant to bacteria and fungus, while also being hypoallergenic and completely free of harmful chemicals. Bamboo is also eco-friendly due to the rapid growth and self-preservation of the bamboo plant, which is so self-sufficient that it requires no pesticides or fertilizer to thrive. Additionally, the wood-based fabric may have a high-degree of biodegradability such that, upon being disposed-of, the wood-based fabric degrades, thereby protecting the environment from long-term waste (which is especially advantageous when carrying highly-concentrated, highly-volatile essential oils, such as with concentrations about 80%, above 90%, above 95%, and even approaching 100%).

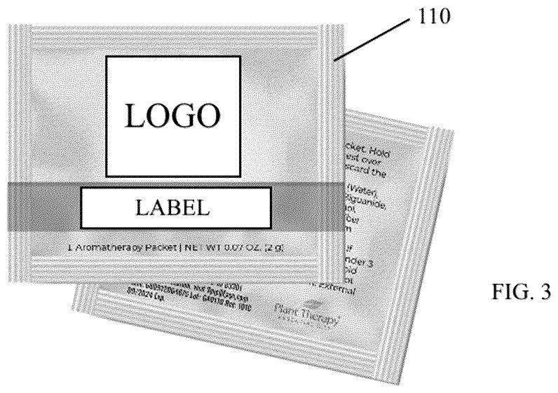

As shown in FIG. 3, the carrier 106 may be packaged individually, meaning the carrier 106 may be pre-filled with the aromatherapy oil at a manufacturing facility (or medical facility, in the case of customized treatment by a medical professional). This contrasts with available products, which are typically filled by the patient once they are “empty” after a previously-filled portion has mostly diffused. Advantageously, this individual packaging provides the ability to use aromatherapy treatments without necessitating filling stations, large quantities of hazardous oils, reliance on precise at-home device fills, and the like. Other advantages may include improved patient safety by eliminating the risk of patient-to-patient contamination. Additionally, when a packet is opened, the aroma will be fresh and vibrant (whereas with other packaging options, the scent will dissipate over time such that freshness may be lost). A new packet for each patient therefore ensures that he or she receive a positive aromatic experience. In certain exemplary embodiments, effective aromatherapy may be achieved for a time period of between about 5 minutes and about 1 hour, such as between about 15 minutes and about 30 minutes, which the inventors have found is long enough for effective treatment but within an appropriate time limit to avoid unwanted exposure (by the patient or others).

The packaging 110 may generally enclose the carrier 106 in an air-tight manner, which may generally increase the life of the carrier 106 as well as prevent unwanted aromas near the carrier 106 prior to breaking the air-tight seal (e.g., during storage or any other time prior to desired use). Optionally, the packaging 110 may be filled with a preservative material, preventing premature degradation of the carrier. The packaging may be formed with any suitable material. For example, the packaging may be formed with a non-degradable material (such as a plastic), or another non-degradable material that is capable of holding an air-tight seal. This is particularly desirable when using concentrated essential oils since essential oils oxidize over time. Such oxidization may reduce the effectiveness of the essential oils, and in some cases may make them harmful.

Since the carrier 106 and packaging 110 generally overcome certain disadvantages of certain products, other characteristics of the aromatherapy product can be adjusted for optimal treatment. For example, given the individual and disposable nature of the carrier 106, the aromatherapy oil may be mixed with a relatively-high proportion by mass of essential oil. This is possible since (1) the carrier 106 may be opened for the first time away from other people (e.g. outside a medical facility), and (2) a relatively-high and relatively-quick rate of diffusion is not necessarily undesirable (and in fact may be desirable), since refilling is unnecessary (given that another carrier 106 may be opened when needed).

In some embodiments, the carrier may be marketed within a kit of aromatherapy products. For example, the embodiments above, including individual, portable packaging and less issue with waste, may allow for different aromatherapy scents, perhaps in a desirable order of use (for particular treatment goals), to be packaged together. It is also contemplated that the products may be boxed in relative bulk (e.g., with 10-100 products in one box, or another suitable number), which is not practical with existing aromatherapy products.

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

A first general aspect includes an aromatherapy product having an aromatherapy oil configured for releasing an aroma when exposed to an outside environment. The aromatherapy product also includes a carrier, where the carrier may include an absorption portion for receiving a predetermined amount of the aromatherapy oil, where the absorption portion includes a wood-based fabric.

In certain implementations, the wood-based fabric includes a bamboo. The absorption portion may only be formed of bamboo. In certain implementations, the carrier may include only bamboo.

In certain implementations, the individual package is formed with a plastic.

In certain implementations, the aromatherapy product may include an individual package configured for enclosing the carrier in an air-tight manner.

In certain implementations, the carrier is biodegradable.

In certain implementations, the aromatherapy oil has a mixture may include at least 80 percent of an essential oil by mass.

In certain implementations, the carrier is designed and filled with oil such that aromatherapy is effective for a period of between about 15 minutes and about 30 minutes.

In certain implementations, the aromatherapy product is configured for therapeutic purposes. In other implementations, the aromatherapy product is configured for at least one of a comfort purpose, a cosmetic purpose, and another non-therapeutic purpose.

Another general aspect includes a method for forming an aromatherapy product having any suitable combination of features from the first general aspect.

We claim:

1. An aromatherapy product, comprising:
an aromatherapy oil configured for releasing an aroma when exposed to an outside environment;
a carrier comprising an absorption portion for receiving a predetermined amount of the aromatherapy oil, wherein the absorption portion includes a wood-based fabric.

2. The aromatherapy product of claim 1, wherein the wood-based fabric includes a bamboo.

3. The aromatherapy product of claim 2, wherein the absorption portion consists of bamboo.

4. The aromatherapy product of claim 2, wherein the carrier consists of bamboo.

5. The aromatherapy product of claim 1, further comprising an individual package configured for enclosing the carrier in an air-tight manner.

6. The aromatherapy product of claim 4, wherein the individual package is formed with a plastic.

7. The aromatherapy product of claim 1, wherein the carrier is biodegradable.

8. The aromatherapy product of claim 1, wherein the aromatherapy oil has a mixture comprising at least 80 percent of an essential oil by mass.

9. The aromatherapy product of claim 1, wherein the carrier is designed and filled with oil such that aromatherapy is effective for a period of between about 15 minutes and about 30 minutes.

10. The aromatherapy product of claim 1, wherein the aromatherapy product is configured for therapeutic purposes.

11. The aromatherapy product of claim 1, wherein the aromatherapy product is configured for at least one of a comfort purpose, a cosmetic purpose and another non-therapeutic purpose.

12. A method for forming an aromatherapy product, comprising:
packaging a carrier having an absorption portion for receiving a predetermined amount of an aromatherapy oil, wherein the absorption portion includes a wood-based fabric,
wherein the aromatherapy oil is configured for releasing an aroma when exposed to an outside environment.

13. The method of claim 12, wherein the wood-based fabric includes a bamboo.

14. The method of claim 13, wherein the absorption portion consists of bamboo.

15. The method of claim 13, wherein the carrier consists of bamboo.

16. The method of claim 12, wherein a package for receiving the carrier comprises an individual package configured for enclosing the carrier in an air-tight manner.

17. The method of claim 16, wherein the individual package is formed with a plastic.

18. The method of claim 12, wherein the carrier is biodegradable.

19. The method of claim 12, wherein the aromatherapy oil has a mixture comprising at least 80 percent of an essential oil by mass.

20. The method of claim 12, further comprising filling the carrier with oil such that aromatherapy is effective for a period of between about 15 minutes and about 30 minutes.
